(19) United States
(12) Patent Application Publication
(10) Pub. No.: US 2006/0006339 A1
(43) Pub. Date: Jan. 12, 2006

(54) Radiation sensor device and fluid treatment system containing same

(75) Inventors: Jim Fraser, St. Thomas (CA); Catalina Dragoi, Madison, AL (US); Jennifer Gerardi, St. Thomas (CA); Tanya Molyneux, St. Thomas (CA)

Correspondence Address: KATTEN MUCHIN ROSENMAN LLP, 525 WEST MONROE STREET, CHICAGO, IL, 60661-3693, US

(73) Assignee: Trojan Technologies Inc.

(21) Appl. No.: 11/167,059

(22) Filed: Jun. 27, 2005

Related U.S. Application Data
(60) Provisional application No. 60/583,613, filed on Jun. 30, 2004.

(51) Int. Cl. G01J 1/42

(52) U.S. Cl. 250/372

(57) ABSTRACT

The invention relates to a radiation sensor device comprising a housing, a radiation sensor secured with respect to a first portion of the housing and a heat pipe in thermal communication with the first portion of the housing, the heat pipe being configured to transfer heat from portion of the house to a second portion of the housing remote from the first portion of the housing. The heat pipe may be used advantageously to transport or transfer heat away from the sensor components of the device to an area remote therefrom. The heat pipe can be used to transfer heat at a rate that is thousands of times higher than copper. The radiation sensor device may be used in an ultraviolet radiation fluid treatment system such as an ultraviolet radiation water disinfection system.

### CROSS-REFERENCE TO RELATED APPLICATION

The present application claims the benefit under 35 U.S.C. §119(e) of provisional patent application Ser. No. 60/583,613, filed Jun. 30, 2005, the contents of which are hereby incorporated by reference.

### BACKGROUND OF THE INVENTION

1. Field of the Invention

In one of its aspects, the present invention relates to a radiation sensor device. In another of its aspects, the present invention relates to a fluid treatment system comprising a novel radiation sensor device.

2. Description of the Prior Art

Optical radiation sensors are known and find widespread use in a number of applications. One of the principal applications of optical radiation sensors is in the field of ultraviolet radiation fluid disinfection systems.

It is known that the irradiation of water with ultraviolet light will disinfect the water by inactivation of microorganisms in the water, provided the irradiance and exposure duration are above a minimum “dose” level (often measured in units of microwatt seconds per square centimetre). Ultraviolet water disinfection units such as those commercially available from Trojan Technologies Inc. under the tradenames Trojan UV Max™, Trojan UV Logic™ and Trojan UV Swift™, employ this principle to disinfect water for human consumption. Generally, water to be disinfected passes through a pressurized stainless steel cylinder which is flooded with ultraviolet radiation. Large scale municipal waste water treatment equipment such as that commercially available from Trojan Technologies Inc. under the trade-names UV3000™, UV3000 Plus™ and UV4000™, employ the same principal to disinfect waste water. Generally, the practical applications of these treatment systems relates to submersion of treatment module or system in an open channel wherein the wastewater is exposed to radiation as it flows past the lamps. For further discussion of fluid disinfection systems employing ultraviolet radiation, see any one of the following:
U.S. Pat. No. 4,482,809,
U.S. Pat. No. 4,872,980,
U.S. Pat. No. 5,006,244,
U.S. Pat. No. 5,418,370,
U.S. Pat. No. 5,539,210, and
U.S. Pat. No. Re36,896.
   In recent years, such systems have also been successfully used for other treatment of water—e.g., taste and odour control, TOC (total organic carbon) control and/or ECT (environmental contaminant treatment).

In many applications, it is desirable to monitor the level of ultraviolet radiation present within the water under treatment. In this way, it is possible to assess, on a continuous or semi-continuous basis, the level of ultraviolet radiation, and thus the overall effectiveness and efficiency of the disinfection process.

It is known in the art to monitor the ultraviolet radiation level by deploying one or more passive sensor devices near the operating lamps in specific locations and orientations which are remote from the operating lamps. These passive sensor devices may be photodiodes, photoresistors or other devices that respond to the impingent of the particular radiation wavelength or range of radiation wavelengths of interest by producing a repeatable signal level (in volts or amperes) on output leads.

Conventional ultraviolet disinfection systems often incorporate arrays of lamps immersed in a fluid to be treated. Such an arrangement poses difficulties for mounting sensors to monitor lamp output. The surrounding structure is usually a pressurized vessel or other construction not well suited for insertion of instrumentation. Simply attaching an ultraviolet radiation sensor to the lamp module can impede flow of fluid and act as attachment point for fouling and/or blockage of the ultraviolet radiation use to treat the water. Additionally, for many practical applications, it is necessary to incorporate a special cleaning system for removal of fouling materials from the sensor to avoid conveyance of misleading information about lamp performance.

International Publication Number WO 01/17906 [Pearcey] teaches a radiation source module wherein at least one radiation source and an optical radiation sensor are disposed within a protective sleeve of the module. This arrangement facilitates cleaning of the sensor since it is conventional to use cleaning systems for the purposes of removing fouling materials from the protective sleeve to allow for optimum dosing of radiation—i.e., a separate cleaning system for the sensor is not required. Further, since the optical radiation sensor is disposed within an existing element (the protective sleeve) of the radiation source module, incorporation of the sensor in the module does not result in any additional hydraulic head loss and/or does not create a “catch” for fouling materials.

Conventional ultraviolet disinfection systems incorporate Low Pressure (LP) lamps, amalgam lamps, Low Pressure High Output (LPHO) lamps and/or Medium Pressure (MP) mercury vapour lamps. Typically, the lamps are arranged in an array that generates a radiation field of high intensity. When such a high intensity radiation field is used to treat water having relatively high transmittance, the sensor assembly (or assemblies) used in the fluid treatment system are susceptible to overheating and consequent component degradation or destruction (e.g., degradation and/or destruction of the photodiode and/or other electrical components of the sensor assembly).

For example, conventional reactor designs and lamp arrays can result in increase of temperature of the sensor board and/or photodiode therein to greater than 200° C.

Exposure of the sensor device and/or any of its components to such high temperatures can also affect the sensor output signal which may lead to incorrect measurements and consequential incorrect control of the fluid treatment system. All spectra of the energy radiated by the lamps can potentially be converted into heat on a surface being radiated.

Further, all of these problems have been exacerbated over the recent past due to effort to miniaturize radiation sensor devices so that they have a minimal effect on the hydraulic head of the fluid being treated.

Accordingly, there remains a need in the art for a radiation sensor device which obviates or mitigates the deleterious effect of thermal build up of the sensor device due to exposure to high intensity radiation. This can cause premature sensor device failure and/or reduced service life of the sensor device.

### SUMMARY OF THE INVENTION

It is an object of the present invention to obviate or mitigate at least one of the above-mentioned disadvantages of the prior art.

It is an object of the present invention to provide a novel radiation sensor device which obviates or mitigates at least one of the above-mentioned disadvantages of the prior art.

Accordingly, in one of its aspects, the present invention provides a radiation sensor device comprising: a housing; a radiation sensor secured with respect to a first portion of the housing, the radiation sensor arranged to detect incident radiation; and a heat pipe in thermal communication with the first portion of the housing, the heat pipe being configured to transfer heat from the first portion of the housing to a second portion of the housing remote from the first portion of the housing.

Other aspects of the present invention relate to a fluid treatment system incorporating such a radiation sensor device, and to a method of cooling such a radiation sensor device.

Thus, the present inventors have discovered a novel radiation sensor device comprising a housing, a radiation sensor secured with respect to a first portion of the housing and a heat pipe in thermal communication with the first portion of the housing, the heat pipe being configured to transfer heat from a first portion of the housing to a second portion of the housing remote from the first portion of the housing. The heat pipe may be used advantageously to transport or transfer heat away from the sensor components of the device to an area remote therefrom. The heat pipe can be used to transfer heat at a rate in the order of thousands of times greater than copper.

As is generally known in the art of heat pipes, a heat pipe typically consists of a (vacuum tight) enclosure, a working fluid and, optionally, a wick or capillary structure.

To the knowledge of the present inventors, it is heretofore unknown to utilize a heat pipe to transfer heat from one location to another in a radiation sensor device, particularly when used in an ultraviolet radiation water disinfection system.

In the present radiation sensor device, the heat pipe is in thermal communication with a portion of the housing to which the sensor is secured. The term “thermal communication” is used in a broad sense and includes direct contact between the heat pipe and the radiation sensor or in direct contact between the heat pipe and radiation sensor (e.g., the radiation sensor may be secured to a printed circuit board to which a direct or indirect connection can be made to the heat pipe). Of course, it is preferred to have as direct a connection as possible between the heat pipe and the radiation sensor given the efficiency at which the heat pipe can transfer heat from the latter.

Preferably, the heat pipe is configured so as to transfer heat from the portion of the housing to which the radiation sensor is secured to a remote location which can be inside or outside of the reactor or other structure in which the radiation sensor device is being used. For example, it is especially preferred to have the heat pipe extend from the portion of the housing to which the radiation sensor is secured to a portion of the housing which is outside the fluid treatment area of the reactor or fluid treatment system.

The general operation of heat pipes is known in the art. Thus, a heat pipe operates by transferring heat from an element connected to a distal portion of the heat pipe. The heat transferred to the distal portion of the heat pipe causes evaporation of a fluid (e.g., water, mercury and the like) in an enclosure in the heat pipe to form a vapour. This vapour is then transported to a proximal portion of the heat pipe after which the fluid is condensed to form a liquid in the proximal portion of the heat pipe. During condensation of the liquid, heat is liberated from the proximal portion of the heat pipe. The condensed liquid is then transported back to the distal portion of the heat pipe via a wick or capillary structure in the heat pipe. In some cases, it is possible to eliminate the wick, particularly if the heat pipe is oriented in a substantially vertically thereby allowing gravity to facilitate transport of the condensed liquid back to the distal portion of the heat pipe.

The heat pipe includes a container (or enclosure) to isolate the working fluid (and create a partial internal vacuum) from the outside environment. The selection of the container material depends on factors such as: compatibility with the working fluid and external environment, strength to weight ratio, thermal conductivity, ease of fabrication, porosity and the like.

The selection of the working fluid is conventional. The factors involved in selecting the working fluid include: compatibility with wick and enclosure materials, good thermal stability, wettability of wick and enclosure materials, vapour pressure not too high or low over the operating temperature range, high latent heat, high thermal conductivities, low liquid and vapour viscosities, high surface tension, the operating temperature range and acceptable freezing or pour point.

The wick or capillary structure is a porous structure and can be made of a material such as steel, aluminum, nickel or copper. It is also possible to use so-called metal foams and felts. As stated above, in certain cases, the use of a wick or capillary structure is optional.

In the present radiation sensor device, the heat pipe is used advantageously to transport or transfer heat away from the sensor components of the device to an area remote therefrom. In some embodiments, it is desirable to dissipate the transferred heat from the remote area, for example, by using a reactor wall, air cool fins, active cooling (e.g., water loops around the distal end of the heat pipe) and the like.

In a preferred embodiment of the present invention, the radiation sensor device comprises a radiation detector and a body portion or housing. The radiation detector contains a photodiode or other sensing element which is able to detect and respond to incident radiation. The body portion (or housing) houses one or more of electronic components, mirrors, optical components and the like. The optical radiation sensor is disposed within a protective sleeve. The protective sleeve may comprise first radiation transparent region in substantial alignment with the radiation detector (or sensing element) and a radiation opaque second region which is in substantial alignment with the body portion of the sensor. Those of skill in the art will also appreciate that the sensing element may be protected by its own integral protective. (e.g., quartz) sleeve which may be positioned inside a lamp sleeve, the latter being coated to provide thermal protection.

Throughout this specification, reference is made to a preferred embodiment of the present invention with a protective sleeve containing a “radiation transparent” region and a “radiation opaque” region. Of course, those of skill in the art will recognize that these terms will depend on the nature of radiation present in the radiation field. For example, if the present invention is employed in an ultraviolet (UV) radiation field, it is principally radiation in this portion of the electromagnetic spectrum to which the “radiation opaque” region should be opaque—i.e., the radiation opaque region may be transparent to radiation having characteristics (e.g., wavelength) different than radiation to be blocked. By “radiation opaque” is meant that no more than 5%, preferably no more than 4%, preferably no more than 3%, of the radiation of interest (e.g., this could be radiation at all wavelengths or at selected wavelengths) from the radiation field will pass through the region and impinge on the radiation sensing element. Thus, in some embodiments of the invention, all radiation (e.g., one or more of UV, visible and infrared radiation) present in the radiation field will be blocked to achieve thermal protection of the sensor in addition to eliminating impingement of incident radiation. In other embodiments of the invention, a pre-determined portion of radiation (e.g., one or two of UV, visible and infrared radiation) present in the radiation field will be blocked to achieve thermal protection of the sensor while allowing impingement of a pre-determined portion of incident radiation.

Depending on the radiation field in question, the radiation opaque region may be provided on the protective sleeve in a number of different ways. For example, it is possible to utilize a metallic layer disposed on the interior or exterior of the protective sleeve to confer radiation opacity to the protective sleeve. The metallic layer may compromise at least one member selected from the group comprising stainless steel, titanium, aluminum, gold, silver, platinum, nitinol and mixtures thereof. Alternatively, a ceramic layer may be disposed on the interior or the exterior of the protective sleeve to confer radiation opacity to the protective sleeve. In yet another embodiment, the radiation opaque layer may comprise of porous metal structure and combination with a metal material. The porous metal structure may contain a metal selected from the group of metallic layers referred to above. Examples of non-metal materials in this embodiment of the radiation opaque layer include an elastomer secured to the porous metal structure.

In another embodiment, radiation specific opacity may be conferred to the protective sleeve by placement in the interior or the exterior thereof a filter layer which will exclude deleterious radiation but allow radiation of interest to pass through the protective sleeve to be detected by the sensor. Thus, again using the example of an ultraviolet radiation sensor, in many cases, the wavelength of interest for detection is in the range of from about 210 to about 300 nm. It is possible to utilize a layer made from a filter material which will allow substantially only radiation in this range through the protective sleeve allowing detection of radiation while minimizing or preventing thermal build-up compared to the situation where all radiation from the radiation field is allowed to enter the protective sleeve. Non-limiting examples of suitable such filter materials may be made from heavy metal oxides of varying thickness and/or numbers of layers depending on the type of radiation being sensed. Those of skill in the art will further appreciate that the optical radiation sensor may have a thermal opaque region as well as a filtered region to protect the sensing element (e.g., photodiode) of the optical radiation sensor.

The provision of the radiation transparent region may take a number of forms. This can be achieved by physically placing a metal layer or depositing a metal layer on the interior or exterior of the protective sleeve such that the radiation transparent region has a desired shape. For example, the radiation transparent region may have an annular shape, a non-annular shape, a rectilinear shape, a curvilinear shape, a substantially circular shape and the like. Further, the radiation opaque region may be designed to provide a plurality (i.e., two or more) of radiation transparent regions.

The manner of disposing the radiation opaque region on the protective sleeve is not particularly restricted. For example, the radiation opaque layer may be adhered, mechanically secured or friction fit to the protective sleeve. The latter two approaches work particularly well when the radiation opaque layer is disposed on the exterior of the protective sleeve. For the interior of the protective quartz sleeve, it is possible to insert a split expanding sleeve. The first approach is preferred in the case where the radiation opaque layer is disposed on the interior or exterior of the protective sleeve. This approach may be used to deposit a fully or selective radiation opaque layer, for example, via vapor deposition, electron beam gun deposition or the like of a metal oxide (e.g., silicon dioxide, titanium dioxide, etc.).

### BRIEF DESCRIPTION OF THE DRAWINGS

Embodiments of the present invention will be described with reference to the accompanying drawings, wherein like reference numerals denote like parts, and in which:

FIG. 1 is a cross-sectional view of a preferred embodiment of the present radiation sensor device; and

FIG. 2 is an enlarged portion of Section A in FIG. 1.

### DETAILED DESCRIPTION OF THE PREFERRED EMBODIMENTS

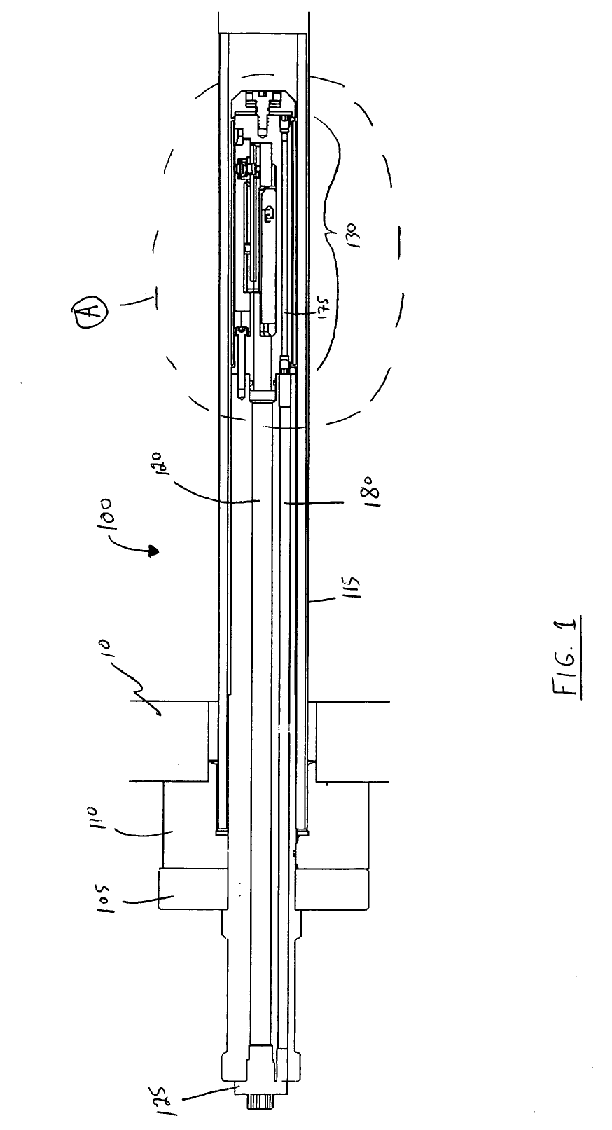

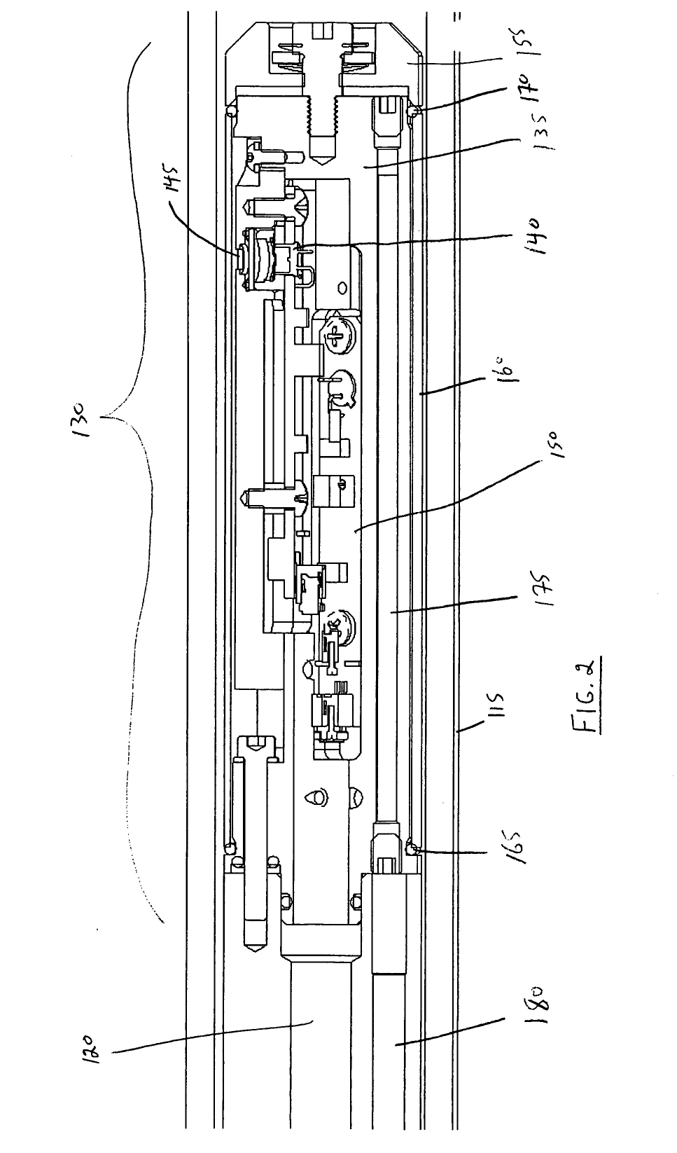

With reference to FIGS. 1 and 2, there is illustrated a radiation sensor device 100. Radiation sensor device 100 is secured to a wall 10 of a reactor such as one described hereinabove. The precise manner in which radiation sensor device 100 may be affixed to wall 10 is not particularly restricted. For example, this can be done through the use of an appropriate combination of mechanical securing elements and O-rings or the like.

Radiation sensor device 100 comprises a gland plate 105 and a transition gland plate 110 both positioned on the exterior of the reactor defined by wall 10.

Radiation sensor device 100 further comprises a protective sleeve 115 which is substantially radiation transparent. Disposed within protective sleeve 115 is a support element 120.

Disposed at a proximal end of support element 120 is an electrical connector 125. Disposed at a distal end of support element 120 is a radiation sensor apparatus 130 which will be described in more detail with reference to FIG. 2.

Radiation sensor apparatus 130 comprises a housing 135 to which a radiation sensor 140 is secured. Radiation sensor 140 may be photodiode, a photoresistor and the like. Housing 135 included a window 145 to allow incident radiation to contact radiation sensor 140. Secured to housing 135 is a printed circuit board 150 containing other components of the radiation sensor apparatus. These other components may include one or more of a signal amplification element, a signal calibration element and a signal transistor element.

Radiation sensor apparatus 130 further includes an end cap 155 and an inner protective sleeve 160 which is sealed to housing 135 via a pair of O-rings 165-170. The provision of inner sleeve 160 and/or end cap 155 is optional.

Also disposed in housing 135 is a first heat pipe 175. First heat pipe 175 is in thermal connection with a second heat pipe 180. Second heat pipe 180 extends to the opposite end of radiation sensor device 100. The construction and operation of heat pipes 175,180 is as discussed above. The thermal connection between heat pipes 175,180 may be direct or indirect.

While heat pipes 175,180 are in a coaxial (e.g., end-on-end) relationship, it is possible to dispose heat pipes 175,180 in a side-by-side or annular relationship (e.g., when three or more heat pipes are used) with respect to housing 135. It is also possible to combine both a coaxial and a side-by-side/annular orientation of heat pipes. It is also possible to have heat pipes overlap axially. It is also possible for the support structure itself to be a heat pipe—i.e., support element 120 could itself be a heat pipe.

While not shown for illustrative purposes, it is possible and, in many cases preferred, to incorporate in protective sleeve 115 a radiation opaque layer such as is described in U.S. patent application Ser. No. 10/845,588 filed May 14, 2004 [Verdun et al.]. In this preferred embodiment, it is possible to have the radiation opaque layer extend from wall 10 to a point just proximal of window 145 of radiation sensor apparatus 130.

In operation, as heat is built up on radiation sensor 140 and/or printed circuit board 150, such heat is transferred to heat pipe 175 which transfers the heat to heat pipe 180 and away from radiation sensor apparatus 130.

It is possible and, in many cases preferred, to incorporate the present radiation sensor device with an annular arrangement of radiation sensor modules as described in the U.S. provisional patent application Ser. No. 60/583,614 filed on Jun. 30, 2004 (Gowlings Ref: T8468253US).

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A radiation sensor device comprising: a housing; a radiation sensor secured with respect to a first portion of the housing, the radiation sensor arranged to detect incident radiation; and a heat pipe in thermal communication with the first portion of the housing, the heat pipe being configured to transfer heat from the first portion of the housing to a second portion of the housing remote from the first portion of the housing.

2. The radiation sensor device defined in claim 1, comprising a plurality of heat pipes in thermal communication with the first portion of the housing, each heat pipe being configured to transfer heat from the first portion of the housing to the second portion of the housing.

3. The radiation sensor device defined in claim 2, wherein the plurality of heat pipes are arranged in a substantially coaxial manner.

4. The radiation sensor device defined in claim 2, wherein the plurality of heat pipes are arranged in a substantially non-coaxial manner.

5. The radiation sensor device defined in claims 2, comprising a first plurality of heat pipes arranged in a substantially coaxial manner and a second plurality of heat pipes arranged in a substantially non-coaxial manner.

6. The radiation sensor device defined in claim 1, wherein the first portion of the housing comprises one or more of the following components: a signal amplification element, a signal calibration element and signal transmitter element.

7. The radiation sensor device defined in claims 1, wherein the first portion of the housing comprises a printed circuit board on which the radiation sensor and one or more of the following components is secured: a signal amplification element, a signal calibration element and signal transmitter element.

8. The radiation sensor device defined in claim 1, further comprising a protective sleeve substantially encompassing the first portion of the housing, the protective sleeve comprises a radiation transparent first region and a radiation opaque second region, the radiation transparent first region being oriented to include the radiation sensor.

9. The radiation sensor device defined in claim 8, wherein the radiation opaque layer comprises a metallic layer.

10. The radiation sensor device defined in claim 9, wherein the metallic layer comprises at least one member selected from the group comprising stainless steel, titanium, aluminum, gold, silver, nickel, platinum, nitinol and mixtures thereof.

11. The radiation sensor device defined in claim 1, wherein the housing comprising a plurality of radiation sensors arranged annularly with respect to a longitudinal axis of the housing.

12. The radiation sensor device defined in claim 1, further comprising a mounting element to secure the radiation sensor device in a fluid treatment system.

13. The radiation sensor device defined in claim 1, further comprising a mounting element to secure the radiation sensor device in a cantilevered manner in a fluid treatment system.

14. A fluid treatment system comprising a fluid treatment zone having disposed therein at least one radiation source and the radiation sensor device defined in claim 1.

15. A fluid treatment system comprising a fluid treatment zone having disposed therein a plurality of radiation sources and the radiation sensor device defined in claim 1.

16. The fluid treatment system defined in claim 14, wherein the radiation sensor device comprises a plurality of radiation sensors.

17. The fluid treatment system defined in claim 16, wherein the ratio of radiation sources to radiation sensors is 1:1.

18. The fluid treatment system defined in claim 14, wherein the ratio of radiation sources to radiation sensors is greater than 1:1.

19. The fluid treatment system defined in claim 16, wherein the plurality of radiation sources is arranged annularly with respect to the radiation sensor device.

20. A method of cooling the radiation sensor device defined in claim 1, the method comprising the steps of:
(i) transferring heat from the first portion of the housing to a distal portion of the heat pipe;
(ii) evaporating a fluid contained in the heat pipe to form a vapour;
(iii) transporting the vapour to a proximal portion of the heat pipe;
(iv) condensing the fluid to form a liquid in the proximal portion of the heat pipe;
(v) transferring heat generated in Step (iv) from the proximal portion of the heat pipe to the second portion; and
(vi) transporting liquid condensed in Step (iv) to the distal portion of the heat pipe via a capillary structure contained in the heat pipe.

21. The method defined in claim 20, wherein Steps (i)-(vi) are sequentially repeated.
